(19) United States
(12) Patent Application Publication
(10) Pub. No.: US 2026/0117245 A1
(43) Pub. Date: Apr. 30, 2026

(54) Wheat leaf rust resistance protein and encoding gene and use thereof

(71) Applicants: PEKING UNIVERSITY INSTITUTE OF ADVANCED AGRICULTURAL SCIENCES; SHANDONG LABORATORY OF ADVANCED AGRICULTURAL SCIENCES IN WEIFANG, Weifang, Shandong (CN)

(72) Inventors: Shisheng CHEN, Weifang, Shandong (CN); Hongna LI, Weifang, Shandong (CN); Lei HUA, Weifang, Shandong (CN); Xiaodong WANG, Weifang, Shandong (CN); Rui SONG, Weifang, Shandong (CN); Yanna LIU, Weifang, Shandong (CN)

(73) Assignees: PEKING UNIVERSITY INSTITUTE OF ADVANCED AGRICULTURAL SCIENCES, Weifang, Shandong (CN); SHANDONG LABORATORY OF ADVANCED AGRICULTURAL SCIENCES IN WEIFANG, Weifang, Shandong (CN)

(21) Appl. No.: 19/119,040

(22) PCT Filed: Jun. 29, 2023
(86) PCT No.: PCT/CN2023/104202

§ 371 Date: Apr. 7, 2025

(30) Foreign Application Priority Data
Oct. 12, 2022 (CN) 202211247709.2

(51) Int. Cl. C12N 15/82; C07K 14/415

(52) U.S. Cl. CPC C12N 15/8282; C07K 14/415; C12N 15/8205

(57) ABSTRACT

A wheat leaf rust resistance protein, its encoding gene, and the use thereof are provided. The wheat leaf rust resistance protein includes any of the following (a)-(c): (a) a protein, which consists of the amino acid sequence represented by SEQ ID NO: 3; or (b) a protein, which has an amino acid sequence obtained after the amino acid sequence of the protein (a) undergoes substitution and/or deletion and/or addition of one or more amino acids and which has anti-wheat-leaf-rust activity; or (c) a protein, which has 80% or more identity with the amino acid sequence defined in any of (a) and (b) and which has the same function.

### CROSS-REFERENCE TO RELATED APPLICATIONS

The present application is a National Stage of International Patent Application No. PCT/CN2023/104202, filed on Jun. 29, 2023, and is based upon and claims the benefit of priority on the basis of Chinese Patent Application No. 202211247709.2, filed on Oct. 12, 2022, the application of which is incorporated herein in its entirety by reference.

### INCORPORATION BY REFERENCE OF SEQUENCE LISTING

The instant application includes a Sequence Listing that has been submitted electronically in XML format and is hereby incorporated by reference in its entirety. The said XML file, named 44887_SequenceListing.xml, is 18,729 bytes in size. The sequence listing includes 7 sequences with SEQ ID NOs: 1 to 7, which are substantially identical in substance to the sequences disclosed in the PCT application. The sequence listing does not include any new matter.

### TECHNICAL FIELD

The present application relates to the field of wheat breeding, and in particular, to a wheat leaf rust resistance protein and an encoding gene and use thereof.

### BACKGROUND

Wheat is a worldwide food crop, serving as a staple food to approximately one third of the global population. However, the safe production of wheat is threatened by various fungal diseases, including wheat leaf rust. Wheat leaf rust, caused by the fungal pathogen Puccinia triticina (Pt), is an airborne fungal disease characterized by its wide distribution, rapid transmission, significant damage, and yield loss. This disease is prevalent in major wheat producing regions worldwide, such as Europe, North America, Asia, Australia, and Africa. It primarily infects wheat leaves, disrupting photosynthesis, which leads to yield reductions typically ranging from 5% to 15% and exceeding 40% in severe cases. Therefore, controlling wheat leaf rust is a critical task in wheat production.

Cloning and utilizing wheat leaf rust resistance genes to develop resistant wheat varieties is the most economical, safe, and effective method for preventing and treating this disease. The wheat leaf rust resistance gene Lr47, introgressed from the wild relative Aegilops speltoides (SS), has demonstrated near-immunity and broad-spectrum resistance against Pt pathotypes in multiple countries worldwide. Therefore, once cloned and transferred into susceptible wheat, Lr47 will have great application prospects in wheat leaf rust resistance breeding.

To date, approximately 82 wheat leaf rust resistance genes (Lr1-Lr82) have been formally named internationally. However, due to the large genome size and high proportion of repetitive sequences (over 80%) in wheat, the isolation and cloning of functional genes in wheat has lagged behind those in other crops such as rice and maize. Currently, only a few number of wheat leaf rust resistance genes have been successfully isolated and cloned.

### SUMMARY

The main objective of the present application is to provide a wheat leaf rust resistance protein, its encoding gene, and use thereof, to address the problem of wheat susceptibility to leaf rust and resulting yield losses in the prior art.

In order to achieve the described objective, a first aspect of the present application provided is a wheat leaf rust resistance protein represented by any of (a)-(c): (a) a protein having an amino acid sequence as shown in SEQ ID NO: 3; or (b) a protein derived from amino acid sequence of (a) by substituting and/or deleting and/or addition of one or more amino acids and which has retain resistance to wheat leaf rust; or (c) a protein having 80% or more identity with the amino acid sequence as defined in any of (a) or (b) and which retain the same function.

Further, provided is a protein having at least 85% or more sequence identity, preferably 90% or more, more preferably 95% or more, and even more preferably 99% or more, with the amino acid sequence defined in any of (a) and (b) and which retain the same function.

In order to achieve the described objective, according to a second aspect of the present application provides a wheat leaf rust resistance gene, which is as shown in any of (i)-(iv): (i) a nucleotide sequence encoding the wheat leaf rust resistance protein; or (ii) a nucleotide sequence that hybridizes under stringent conditions to the DNA molecule defined in (i) and encodes the wheat leaf rust resistance protein as described in claim 1; or (iii) a nucleotide sequence as shown in SEQ ID NO: 2; or (iv) a gene, which has 70% or more identity with any of the nucleotide sequences defined in (i) to (iii) and encodes a protein having the same function.

Further, provided is a gene which has 75% or more, preferably 85% or more, more preferably 95% or more, and even more preferably 99% or more identity with any of the nucleotide sequences defined in (i) to (iii) and encodes a protein having the same function.

In order to achieve the described objective, according to a third aspect of the present application, an expression cassette is provided, comprising a regulatory sequence and the wheat leaf rust resistance gene.

Further, the regulatory sequence comprises a promoter. Preferably, the promoter comprises one or more of the following: constitutive promoters, enhanced promoters, tissue-specific promoters, or inducible promoters.

In order to achieve the described objective, according to a fourth aspect of the present application, a recombinant vector is provided, comprising the wheat leaf rust resistance gene or the expression cassette.

Further, the recombinant vector comprises a translational control signal; preferably, the translational control signal comprises an enhancer; preferably, the enhancer comprises a translation enhancer and/or a transcriptional enhancer; preferably, the translational control signal is derived from a natural sequence or an artificially synthesized sequence; preferably, the recombinant vector comprises a plant expression vector; preferably, the plant expression vector comprises a binary vector for Agrobacterium-mediated transformation or a vector for gene gun bombardment; preferably, the plant expression vector comprises pCAMBIA1300; preferably, the recombinant vector comprises a reporter gene; preferably, the reporter gene comprises a resistance gene, or a gene expressing an enzyme or a luminescent compound that produces a color change; and preferably, the resistance gene comprises an antibiotic resistance gene or a herbicide agent resistance gene.

In order to achieve the described objective, according to a fifth aspect of the present application, a host cell transformed with the recombinant vector is provided; preferably, the host cell is a non-plant host cell; preferably, the host cell comprises Escherichia coli or Agrobacterium tumefaciens; preferably, the Escherichia coli comprises DH5a; and preferably, the Agrobacterium tumefaciens strain comprises EHA105.

Provided is the application of the wheat leaf rust resistance protein, or the wheat leaf rust resistance gene, or the expression cassette, or the recombinant vector, or the host cell in regulating the resistance of a plant to leaf rust, enhancing or reducing the resistance of a plant to leaf rust, or breeding a transgenic plant with enhanced or reduced resistance to leaf rust, or wheat leaf rust resistance breeding.

In order to achieve the described objective, according to a sixth aspect of the present application, provides a method for preparing a transgenic plant, comprising introducing the wheat leaf rust resistance gene, or the expression cassette, or the recombinant vector, or the host cell into a target plant, so as to obtain the transgenic plant with resistance to leaf rust.

Further, the recombinant vector is introduced into a target plant by means of a plant virus vector, a gene gun, or Agrobacterium-mediated transformation; preferably, the target plant is a dicotyledonous plant or a monocotyledonous plant; preferably, the target plant is wheat; preferably, the wheat plant is Fielder wheat; and preferably, the wheat leaf rust resistance gene is driven by a constitutive promoter.

In order to achieve the described objective, according to a seventh aspect of the present application, provided is a breeding method for enhancing or reducing the resistance of a plant to leaf rust. The breeding method comprises enhancing or reducing the activity or content of the wheat leaf rust resistance protein in a target plant to enhance or reduce the resistance of the plant to leaf rust.

Further, the target plant is a dicotyledonous or monocotyledonous plant; preferably, the target plant is wheat; preferably, the wheat is Fielder wheat; preferably, the leaf rust disease is caused by a Pt pathotype; preferably, the pathotype of Pt is one prevalent in China, and the pathotypes of Pt in China include, but are not limited to, FHJL, PHOS, FHJR, THDB, PHRT, PHTT, HCJR, or FHHM.

By applying the technical solution of the present application, a novel leaf rust resistance protein and its encoding gene, as well as their thereof are provided. These advancements facilitate the study of the disease resistance mechanism of a resistance gene against pathogenic bacteria and improve the resistance of wheat to leaf rust, and provide a reliable and effective source of leaf rust resistance for wheat molecular breeding, and have a great application and promotion value for wheat leaf rust resistance breeding.

### BRIEF DESCRIPTION OF THE DRAWINGS

The accompanying drawings, which form a part of the present application, are used to provide a further understanding of the present application. The schematic embodiments of the present application and the description thereof are used to explain the present application, and do not form improper limits to the present application. In the drawings:

FIG. 1 shows a graph of the phenotypic results of near-isogenic lines with and without Lr47 inoculated with a physiological pathotype of leaf rust as described in Example 1 of the present application.

FIG. 2 shows a schematic diagram of the fine mapping of the leaf rust resistance gene Lr47 as described in Example 2 of the present application. (a) in FIG. 2 is a schematic diagram of 7A chromosome of wheat line Kern Lr47; (b) in FIG. 2 is a schematic diagram of a genome-specific molecular marker developed on an exogenous 7S chromosome, and with the physical position is referred to Triticum aestivum cv. Chinese Spring v1.0 reference genome; (c) in FIG. 2 is a schematic diagram of a key recombinant events occurring between 67.6-85.2 megabase (Mb) obtained with wheat CSph1b to induce partial homologous chromosome recombination of the introgressed 7S chromosome; (d) in FIG. 2 is a schematic diagram of a linkage genetic map of for Lr47 fine mapping using a segregating population constructed using susceptible ethyl methane sulfonate (EMS) mutant m118 and wild-type Kern Lr47; and (e) in FIG. 2 is a schematic diagram of candidate genes located within the candidate chromosomal interval in the Aegilops speltoides TS01 reference genome of 1.

FIG. 3 shows a graph of validation of the Lr47 candidate gene using EMS mutants as described in Example 3 of the present application. (a) in FIG. 3 is a graph of infection types of a susceptible mutant inoculated with Pt pathotype THDB; and (b) in FIG. 3 is a schematic diagram of the gene structure of the Lr47 candidate gene and an EMS-induced susceptible mutant that underwent base/amino acid changes.

FIG. 4 shows a graph of transgenic complementary verification results of the Lr47 candidate gene as described in Example 4 of the present application. (a) in FIG. 4 is a schematic diagram of an Lr47 genome segment used for transgenic complementary verification, including 2,097 bp upstream of a start codon, 3,132 bp of the full gene length (from ATG to TGA), and 2,005 bp downstream of the gene; and (b) in FIG. 4 is a graph of the differential phenotype results of the control variety Fielder and some Ti transgenic plants 10 days after inoculation with the Pt pathotype PHQS.

### DETAILED DESCRIPTION OF THE EMBODIMENTS

It is noted that the embodiments of the present application and the characteristics in the examples can be combined, provided there are no conflicts. Hereinafter, the present application will be described in detail with reference to examples.

### Definition of Terms

Translational control signals, also known as protein translational control signals, refer to nucleotide sequences, including, but not limited to enhancers, that are present upstream and/or downstream of a gene and can regulate the translation of encoded protein, often by influencing the transcription of a target gene.

As mentioned in the background, with climatic changes and the continuous emergence of new virulent Pt pathotypes, leaf rust resistance genes in wheat varieties are increasingly difficult to develop resistance to these new virulent pathotypes, resulting in the loss of leaf rust resistance in wheat. Once a virulent pathotype becomes prevalent, it will cause serious harm and seriously threaten the safe production of wheat. Using a leaf rust resistance gene to breed novel disease-resistant wheat varieties is the most economical and effective way to control the disease. Presently, although there are over 80 formally-named wheat leaf rust resistance genes, only a few of them have been successfully isolated and cloned. Accordingly, the inventors of the present application conducted an in-depth study on the leaf rust resistance gene Lr47 from Ae. speltoides, a close relative of wheat, and completed the fine mapping, isolation, cloning, and functional verification of Lr47. It has been found that the resistance protein encoded by Lr47 has the activity of resisting wheat leaf rust. On this basis, the present application proposed a series of protection solutions.

In a first typical embodiment of the present application, provided is a wheat leaf rust resistance protein represented by any of (a)-(c): (a) a protein having an amino acid sequence as shown in SEQ ID NO: 3; or (b) a protein derived from (a) by substituting and/or deleting and/or adding one or more amino acids in the amino acid sequence and which has resistance against wheat leaf rust (anti-wheat-leaf-rust activity); or (c) a protein having 80% or more sequence identity with the amino acid sequence as defined in any of (a) and (b) and having the same function.

The wheat leaf rust resistance protein has resistance against wheat leaf rust. The protein can be mutated/modified by substitution and/or deletion and/or addition of one or more amino acids to the sequence shown in (a). If the mutation occurs at an active site of the protein, may lead to changes in key amino acid binding sites of the protein and affect the anti-wheat leaf rust activity of the protein, leading to an enhancement or reduction or even loss of activity. If the mutation occurs at an inactive site of the protein, it may affect the properties of the protein, such as folding manner and three-dimensional structure, thereby affecting the physicochemical properties and activity of the protein. Proteins having 80%, 85%, 90%, 95%, 99%, or greater sequence identity and retaining the same function likely share similar active sites, active pockets, and active mechanisms with the protein provided by the sequence (a), are homologous proteins obtained through amino acid mutations. The description of “the same function” as used herein with respect to homologous proteins in the present application, refers to the resistance against wheat leaf rust.

Proteins having the same function can be identified using standard screening assays commonly used by a person skilled in the art.

“Identity,” as used herein, refers to sequence similarity or identity, and in particular, to sequence identity. “Amino acid sequence identity” refers to identity across the entire amino acid sequence. The “identity” (refers to the degree of similarity between two sequences based on the exact matching of their elements, i.e., amino acids) between amino acid sequences is determined by calculating the percentage of identical amino acid residues. “Similarity” (refers to the alignment of sequences where the amino acids are not necessarily identical but are chemically or structurally similar) between amino acid sequences encompasses both identical amino acid residues and amino acid residues with similar side-chain properties. Sequence identity can be determined using alignment programs such as BLAST (Basic Local Alignment Search Tool) or FASTA or like alignment programs.

As used herein, amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

With regard to rules such as substitution and replacement, in general, the effects after replacement of amino acids having similar properties are also similar. For example, in the homologous proteins described above, conservative amino acid substitutions may occur. “Conservative amino acid substitutions” include, but are not limited to:
Substitution of one hydrophobic amino acid (Ala, Cys, Gly, Pro, Met, Val, Ile, or Leu) for another hydrophobic amino acid;
Substitution of one hydrophobic amino acid with a large side chain (Phe, Tyr, or Trp) for another hydrophobic amino acid with a large side chain;
Substitution of one positively charged amino acid (Arg, His, or Lys) for another positively charged amino acid; and
Substitution of one uncharged polar amino acid (Ser, Thr, Asn, or Gln) for another uncharged polar amino acid.

A person skilled in the art is familiar with amino acid substitution rules and would also be able to perform conservative replacements on amino acids according to amino acid replacement rules, such as those known in the prior art, including the “blosum62 scoring matrix”.

In a preferred example, provided is a protein having 85% or more, preferably 90% or more, more preferably 95% or more, and even more preferably 99% or more identity with the amino acid sequence as defined in any of (a) and (b) and having the same function.

The protein variant having identity has resistance against wheat leaf rust similar to or the same as that of the protein as shown in SEQ ID NO: 3.

In a second exemplary embodiment of the present application, a wheat leaf rust resistance gene is provided, as shown in any of (i)-(iv): (i) a nucleotide sequence encoding the wheat leaf rust resistance protein; or (ii) a nucleotide sequence that hybridizes under stringent conditions to the DNA molecule defined in (i) and encodes the wheat leaf rust resistance protein; or (iii) the nucleotide sequence as shown in SEQ ID NO: 2; or (iv) a gene having 70% or more identity with any of the nucleotide sequences defined in (i)-(iii) and encodes a protein having the same function.

The term “DNA molecule hybridization under stringent conditions” as used herein refers to conditions under which a nucleotide sequence specifically hybridizes to a target sequence in a quantity detectably stronger than the non-specific hybridization. Stringent conditions may include, for example, low salt and/or high temperature conditions, such as those provided by about 0.02 M to 0.1 M NaCl (or equivalent salts) at temperatures of about 50° C. to 70° C.

In a preferred embodiment, provided is a gene having 75% or more, preferably 85% or more, more preferably 95%, or more, and even more preferably 99% or more identity with any of the nucleotide sequences defined in (i)-(iii) and encodes a protein having the same function.

The wheat leaf rust resistance gene encodes a protein that confers resistance against wheat leaf rust. A nucleotide is mutated on the basis of the sequence of (i) and hybridizes under stringent conditions to the DNA molecule defined in (i) without a frameshift mutation. If the mutation occurs in the nucleotide encoding an active site of the protein, it may cause key amino acid binding sites of the encoded protein to change, affecting the anti-wheat leaf rust activity of the protein encoded by the gene, leading to an enhanced or reduced or even loss of the activity. If the mutation occurs in a nucleotide encoding an inactive site of the protein, it may affect the folding mode, three-dimensional structure, and other properties of the encoded protein, thereby affecting the physicochemical properties and activity of the protein. Wheat leaf rust resistance genes having 70%, 75%, 85%, 95% or more identity and encoding proteins having the same function, whose active sites, active pockets, active mechanisms, etc. of the proteins encoded by them are most likely the same as the gene provided by the (i) sequence, are homologous genes obtained through nucleotide mutations.

The disease-resistant parent of the present application is Kern Lr47 (PI 638739), which carries an about 150 Mb introgressed chromosomal segment from the wheat wild relatives Ae. speltoides. This segment is translocated to the 7A chromosome of common wheat cultivar Kern. Studies have revealed that this introgression segment harbors a broad-spectrum leaf rust resistance gene, Lr47, which confers strong resistance to a wide range of Pt isolates worldwide. However, due to the complexity of the wheat genome and the lack of genetic research and genome sequence information, the Lr47 gene has not been isolated and cloned previously. In the present application, Lr47 is isolated and cloned through fine mapping using a large segregating population, combined with the MutRNASeq method. Furthermore, its function was verified through independent EMS-induced susceptible mutants and transgenic complementary experiments.

Throughout the present application, the proteins and genes discussed include naturally occurring proteins and genes, as well as isolated proteins and isolated genes. Such an isolated gene can be used to encode a protein conferring resistance to wheat leaf rust and can be used in breeding programs, such as regulating the resistance against wheat leaf rust.

In a third typical embodiment of the present application, provided is an expression cassette, the expression cassette comprising a regulatory sequence and the wheat leaf rust resistance gene.

In the cassette, the regulatory sequences include, but are not limited to, a promoter. Preferably, the promoter includes, but is not limited to, one or more of the following promoters: constitutive promoters, enhanced promoters, tissue-specific promoters, or inducible promoters.

The expression cassette, also known as gene expression cassette, consists of the regulatory sequence and the wheat leaf rust resistance gene, and may further comprise other nucleic acid fragments. The regulatory sequence exerts influence on the expression of transcription, translation, etc., and/or other aspects of the expression of the wheat leaf rust resistance gene. The regulatory sequence can be a nucleic acid fragment such as a promoter, an enhancer, a silencer, or a regulatory protein binding site, wherein the promoter can function in combination with one or more of a constitutive promoter, an enhanced promoter, a tissue-specific promoter, an inducible promoter or other types of promoters to achieve the purpose of regulating gene expression.

In a fourth typical embodiment of the present application, provided is a recombinant vector comprising the wheat leaf rust resistance gene or the expression cassette.

In a preferred embodiment, preferably, the recombinant vector comprises a translational control signal; preferably, the translational control signal comprises an enhancer; preferably, the enhancer comprises a translation enhancer and/or a transcriptional enhancer; preferably, the translational control signal is derived from a natural sequence or an artificial synthetic sequence; preferably, the recombinant vector comprises a plant expression vector; preferably, the plant expression vector comprises a binary vector for Agrobacterium-mediated transformation or a vector for gene gun bombardment; preferably, the plant expression vector comprises pCAMBIA1300; preferably, the recombinant vector comprises a reporter gene; preferably, the reporter gene comprises a resistance gene, or a gene expressing an enzyme or a luminescent compound producing a color change; and preferably, the resistance gene comprises an antibiotic resistance gene or a chemical agent resistance gene.

The recombinant vector comprises a wheat leaf rust resistance gene or the expression cassette, and may also comprise other nucleic acid fragments such as a replication initiation site, a multiple cloning site, and/or a translational control signal, etc. The translational control signal which may be derived from a natural sequence or a synthetic sequence comprises an enhancer, a chaperone, and/or other nucleotide sequence that can affect protein translation. The enhancer may comprise a translation enhancer, and/or a transcriptional enhancer, which can be used independently or in combination to play a role in regulating the transcription and translation of a protein. The recombinant vector can be a plant expression vector and can be transformed into a plant to express a target gene in the plant and produce a target protein so as to play a role. The plant expression vector includes, but is not limited to, a binary vector for Agrobacterium-mediated transformation or a vector for gene gun bombardment. Different transformation methods can be used to introduce the vector into plant cells to improve transformation efficiency. The plant expression vector includes, but is not limited to, pCAMBIA1300 used in the examples.

The recombinant vector may further comprise a reporter gene. Preferably, the reporter gene includes, but is not limited to, a resistance gene or a gene expressing an enzyme or a luminescent compound producing a color change. This allows for determining whether the recombinant vector has been successfully transformed and expressed by various methods such as resistance screening, color screening, and fluorescence screening. The resistance gene includes, but is not limited to, an antibiotic resistance gene or a chemical reagent resistance gene. A transformed parent can be efficiently screened with drugs such as an antibiotic and a chemical reagent, so as to determine whether the recombinant vector is successfully transformed and expressed. Alternatively, transformation success can be assessed phenotypically, without a reporter gene, to address transgenic safety concerns.

In a fifth typical embodiment of the present application, provided is a non-plant host cell, wherein the host cell is transformed with the recombinant vector; preferably, the host cell includes, but is not limited to Escherichia coli or Agrobacterium tumefaciens; preferably, the Escherichia coli includes, but is not limited to, DH5a; and preferably, the Agrobacterium tumefaciens includes, but is not limited to, EHA105.

The host cell is transformed with a recombinant vector, and the recombinant vector can be carried for various purposes, such as recombinant vector copy/replication, gene expression, and gene integration into the host cell chromosome. The host cell may be various strains such as Escherichia coli and Agrobacterium tumefaciens, wherein Escherichia coli may commonly use DH5a, and Agrobacterium tumefaciens may commonly use EHA105.

In a sixth typical embodiment of the present application, provided is an application of the wheat leaf rust resistance protein, or the wheat leaf rust resistance gene, or the expression cassette, or the recombinant vector, or the host cell for use in regulating the resistance of a plant to leaf rust, enhancing or reducing the resistance of a plant to leaf rust, or culturing/producing a transgenic plant having enhanced or reduced resistance to leaf rust, or breeding wheat for leaf rust resistance.

In the application, the wheat leaf rust resistance protein, the gene, the expression cassette, the recombinant vector, or the host cell is employed to regulate the resistance of a plant to leaf rust through the resistance protein or the protein encoded by the resistance gene, etc. Plant leaf rust can be enhanced or reduced through the use of the regulatory sequence, the translational control signal, and/or components of the expression cassette or recombinant vector. A host cell carrying the recombinant vector can be introduced into a target parent plant with various transformation methods to produce a transgenic plant having enhanced or reduced resistance to leaf rust.

In a seventh typical embodiment of the present application, provided is a method for preparing a transgenic plant, comprising: introducing the wheat leaf rust resistance gene, or the expression cassette, or the recombinant vector, or the host cell into a target plant to produce a transgenic plant resistant to leaf rust.

In a preferred embodiment, the recombinant vector is introduced into the target plant by means of a plant viral vector, a gene gun, or Agrobacterium-mediated transformation. Preferably, the target plant is a dicotyledonous plant or a monocotyledonous plant; preferably, the plant is wheat; preferably, the wheat is Fielder wheat; and preferably, the wheat leaf rust resistance gene is driven by a constitutive promoter.

In the method, the recombinant vector is introduced into the target plant by means of a plant viral vector, a gene gun, or Agrobacterium-mediated transformation. In the method for preparing a transgenic plant, the wheat leaf rust resistance gene, the expression cassette, the recombinant vector, or the host cell is introduced into a target plant with various methods such as a plant virus vector, a gene gun, or Agrobacterium-mediated transformation, so as to obtain a transgenic plant having enhanced resistance to leaf rust. The target plant can be a dicotyledonous or monocotyledonous plant; preferably, the monocotyledonous plant can be wheat, and the variety of wheat includes, but is not limited to, Fielder wheat. The method can modulate the expression of the wheat leaf rust resistance gene, or the activity or translation of the encoded protein for example, establishing a mutant library, obtaining a mutant family, or inducing mutations at specific nucleotide sites within the resistance gene, thereby obtaining a transgenic plant having reduced or enhanced resistance to leaf rust.

In an eighth typical embodiment of the present application, provided is a breeding method for enhancing or reducing the resistance of a plant to leaf rust. The method comprises: enhancing or reducing the activity or content of the wheat leaf rust resistance protein in a target plant, thereby enhancing or reducing the resistance of the plant to leaf rust.

The method can enhance or reduce the activity or content of the wheat leaf rust resistance protein in a target plant by means of methods such as mutation of a nucleotide sequence and/or modification of a regulatory sequence and/or a translational control signal, thereby enhancing or reducing the resistance of the plant to leaf rust.

In a preferred embodiment, the target plant is a dicotyledonous or monocotyledonous plant; preferably, the target plant is wheat; preferably, the wheat is Fielder wheat; preferably, the leaf rust is caused by a Pt pathotype; preferably, the Pt pathotype is a Pt pathotype prevalent in China, and the Pt pathotype prevalent in China includes, but is not limited to, FHJL, PHQS, FHJR, THDB, PHRT, PHTT, THTT, HCJR, or FHHM.

The beneficial effects of the present application will be further explained in detail below in conjunction with specific examples.

### Example 1: Resistance Spectrum Analysis of Leaf Rust Resistance Gene Lr47

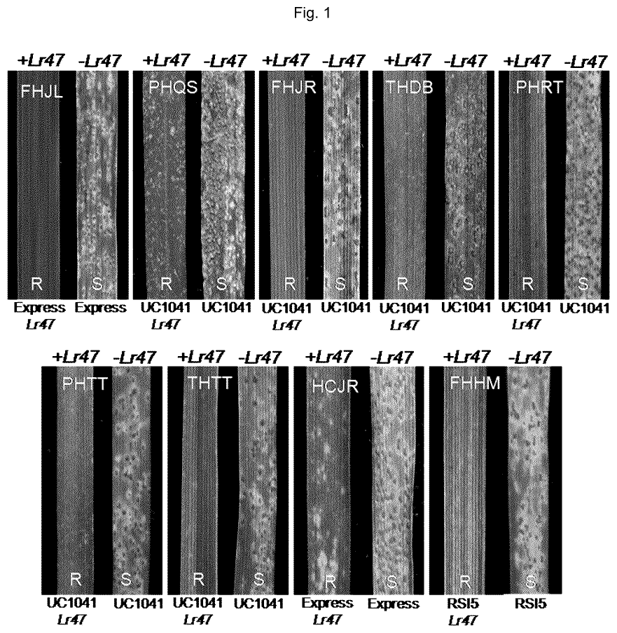

American common wheat lines UC1041, Express, RSI5, and near-isogenic lines UC1041 Lr47, Express Lr47, and RSI5 Lr47 carrying the Lr47 gene were planted in a growth chamber. The growth chamber was maintained under the following conditions: 22° C. during the day, 20° C. at night, 16 h of light, 8 h of darkness, and 80-90% humidity. When wheat seedlings reached the two-leaf-one-heart stage, they were inoculated with nine different Pt pathotypes: FHJL, PHOS, FHJR, THDB, PHRT, PHTT, THTT, HCJR, and FHHM, using a manual sweeping inoculation method. After inoculation, the plants were kept in darkness for 24 hours, followed by at least 2 hours of light. The growth chamber was then set to a normal light cycle. About 10 days after inoculation, the wheat lines were assessed for leaf rust resistance. The leaf rust phenotype was scored according to a 0-4 grading scale (0=immune; 0; =nearly immune; 1=highly resistant; 2=moderately resistant; 3=moderately susceptible; and 4=highly susceptible). As shown in FIG. 1, near-isogenic lines containing the leaf rust resistance gene Lr47 showed nearly immune level resistance (R), while the background material lacking Lr47 was susceptible(S).

### Example 2: Fine Mapping of Leaf Rust Resistance Gene Lr47

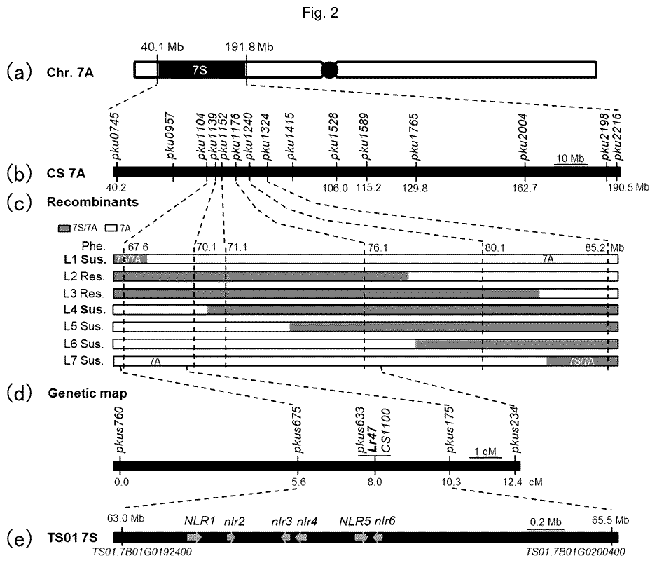

An alien 7S chromosome segment carrying Lr47 (as shown in (a) in FIG. 2) cannot undergo recombination with the 7A chromosome of common wheat. To fine map Lr47, we constructed a segregating population by crossing the resistant parent Kern-Lr47 with the susceptible mutant CSph1b. The ph1b mutant was used to induce recombination between the homoeologous 7S and 7A chromosomes. The specific procedure was as follows: Kern Lr47 was hybridized with susceptible parent CSph1b to produce F₁ progeny using an indoor plant growth chamber. The F₁ plants were self-pollinated to obtain the F₂ population, and then through molecular markers, heterozygous plants that were homozygous for the ph1b gene and carried 7S/7A chromosomes were selected. These selected heterozygous individual plants were then selfed to obtain F₃. As shown in (b) in FIG. 2, we developed 15 genome-specific molecular markers for 7A/7S chromosomes. These molecular markers were used to screen 2,654 F₃ individual plants and obtain individual plants that had undergone recombination within the 150 Mb alien 7S chromosome segment, i.e., recombinants. As shown in (c) in FIG. 2, from the obtained recombinants, we selected plants with a reduced alien chromosome segment genotype of heterozygous 7S/7A and homozygous 7A, which were selected and inoculated with Pt pathotype THDB, for leaf rust phenotypic assessment. By combining genotypic and phenotypic data, Lr47 was mapped between molecular markers pku1104 and pku1152, corresponding to a 3.5 Mb physical interval in the common wheat (CS RefSeq v1.1; (c) in FIG. 2).

Subsequently, the resistant parent Kern Lr47 was crossed with its susceptible EMS-induced mutant line m118 to obtain F₁, which was then self-pollinated to generate an F₂ segregating population. Moreover, Kern Lr47 and m118 were re-sequenced, and bioinformatic analysis was performed to identify single nucleotide polymorphism (SNP) between the parents. By combining these data with the reference genome of Ae. speltoides TS01, CAPS, or sequencing markers were developed. We screened 1,141 F₂ individuals using molecular markers, and considering the phenotypic data from the recombinants, Lr47 was mapped between molecular markers pkus675 and pkus175, co-segregated with molecular markers pkus633 and CS1100 ((d) in FIG. 2). This interval corresponds to a 2.5 Mb physical region in the Ae. speltoides TS01 reference genome that contains a cluster of typical NBS-LRR genes ((e) in FIG. 2).

### Example 3: Susceptible EMS Mutants and Lr47 Candidate Gene Validation

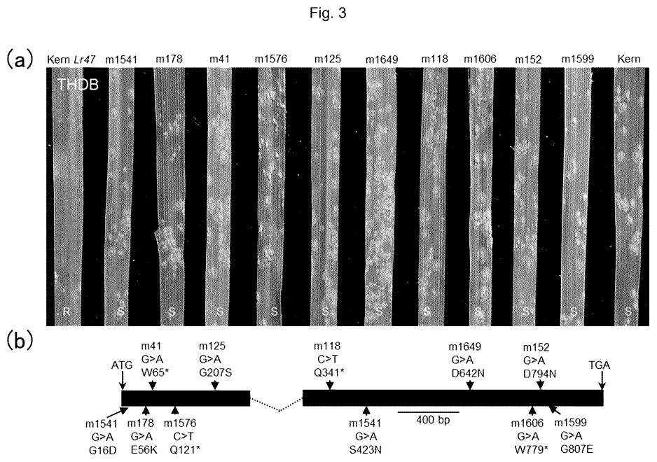

The resistant parent Kern-Lr47 was treated with EMS at a concentration of 0.75% to generate 4,568 independent M₂ mutant families. 562 M₂ mutant families were phenotypically screened in a controlled-environment plant growth chamber. Twenty-five seedlings from each family were inoculated with the Pt pathotype THDB. Ten families were identified that segregated susceptible plants. To avoid seed contamination, the presence of the alien 7S chromosome in susceptible plants was confirmed using molecular markers. Subsequently, susceptible plants were then transplanted, and M₃ seeds were harvested, and phenotypic identification of M₃ individual mutants (including m1541, m178, m41, m1576, m125, m1649, m118, m1606, m152, and m1599) was performed to confirm the susceptible phenotypes of these families ((a) in FIG. 3).

Based on the reference genomes of CS and TS01, we found a cluster of typical NBS-LRR genes within the fine-mapped candidate chromosome interval. This type of gene is the most common disease resistance gene. It is hypothesized that Lr47 may be an NBS-LRR gene. A modified MutRNAseq approach was used to identify the Lr47 candidate gene. RNA-seq of Kern-Lr47 and its recurrent parent Kern were collected after inoculation with Pt pathotype PHQS. De novo assembly was performed to generate transcripts, which were then subjected to local BLASTN analysis and annotation to identify NBS-LRR gene transcripts. A Kern-Lr47 specific NBS-LRR transcript contig (not present in Kern) was identified and used as a reference sequence. Subsequently, ten susceptible mutant families were subjected to RNA sequencing. The results were aligned to the Kern-Lr47 specific NBS-LRR transcript contigs. Analysis revealed that one contig (designated CNL102) contained nonsynonymous mutations in all ten mutant families, including four premature stop codon mutations ((b) in FIG. 3). These results demonstrate that this candidate gene is essential for leaf rust resistance.

### Example 4: Transgenic Complementation Validation of the Lr47 Candidate Gene

To determine whether the candidate gene confers leaf rust resistance, transgenic complementation of the candidate gene was performed.

1. Construction of Complementary Vector p1300-Lr47

To obtain intronic, upstream, and downstream sequences of candidate gene CNL2, we integrated whole-genome resequencing data from Kern Lr47 and mutant line m118 with RNA-seq data from multiple susceptible mutant lines to assemble a genomic segment containing the candidate gene. The accuracy of the obtained sequence was verified through PCR amplification and Sanger sequencing. By combining the genomic and transcript sequences with the analysis of the NCBI database BLASTN/BLASTX, the structure of the gene was determined. The resulting nucleotide sequence as shown in SEQ ID NO: 2 and the amino acid sequence as shown in SEQ ID NO: 3 were obtained, which represent the wheat leaf rust resistance gene (CDS) and a wheat leaf rust resistance protein, respectively.

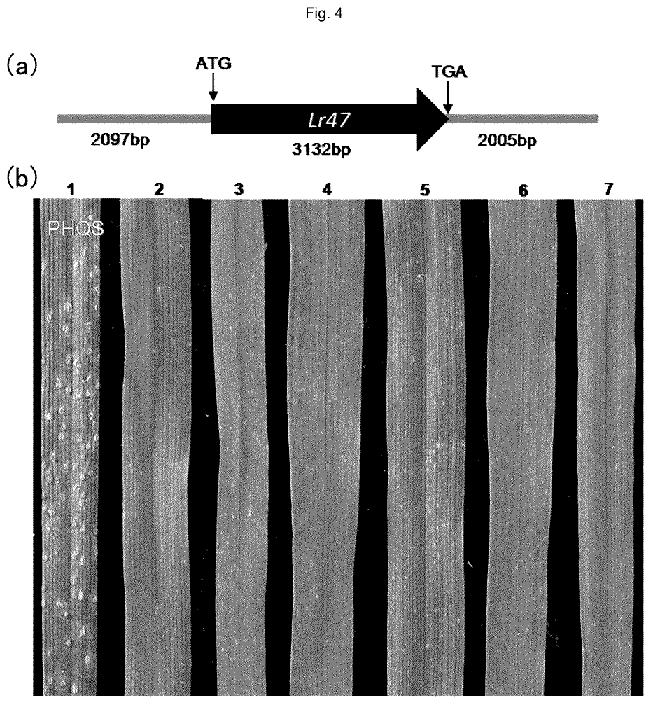

Based on this information, to perform transgenic complementary verification, we constructed an Lr47-containing transgenic vector-amplified genomic fragment as shown in SEQ ID NO: 1, comprising 2,097 bp upstream of the gene start codon, 3,132 bp full length of the gene (from ATG to TGA), and 2,005 bp downstream of the gene, a total genomic sequence of 7,234 bp ((a) in FIG. 4). PCR amplification was performed with primers p1300-Lr47F1 (SEQ ID NO: 4) and p1300-Lr47R1 (SEQ ID NO: 5) as well as p1300-Lr47F2 (SEQ ID NO: 6) and p1300-Lr47R2 (SEQ ID NO: 7). The candidate gene was then recombined onto a linearized pCAMBIA1300 using the In-Fusion HD Cloning Kit (Takara Bio, Beijing) to generate the p1300-Lr47 plasmid.

SEQ ID NO: 1:
cctcggtgaagttggcgtcgaagtgtggctgatggtgcgggtcaggttcatctgtgtggcagtccctaggtgcgggtgaaggccatc
acgacggatctgatccccgcatcgattggtggttgctgcagttcctggtgtgctgaaggtggtggctggaggcgtgcttcctcggcagtgatg
tgtggcttcgactgcaagtggctgcggtgttggtggtgagaggcatctttcggggctggccggaagcttggtggcgtggaaatgtgcaatgc
tacggatgaaaattctgttcggccttggtcggaccggcatcgatggcacccgtgggtgtcattccccttcctggaggcgccgccgagggta
gcgccattgtccacgttgccttgtatcggcaaccatctccggggcgaaagccttgatccgtaggatcggcacgacggcggcgtctttctgat
gttgtttctctgttgggagcttcgtgcttggagataagaggccctatgttgcgctcctccggcgtgcaccgctacccggatcgttcttctctggcg
ctatgtacgatcgtcgctggttctaccaaggaagctggagttgctgttcttcgatgatctccggcgtcggtcgagacgcggcgaggatatcg
gttttgggtaggctcttgtatgtcgtagtgtattgtcgtgtgtggtttcctttgtttgtgtcaggtgtggagttgtgctacgctcgttgttcgcag
cgagaggtagatgtggttgtatggctgtatttctctccttctataaagctaaggtatgcattttgcgtaccctcgaaaaaaacacgttagtggagg
ctgttgctgctaatacgttagtggaggctgctttttgttttctttttttttctttttctttttgagggggttagtggagctgctgctgctaatgcg
cggagagatgcgaggagacgaccgcaagtcgttcattccatttccaggtccacaaatagtttgtttccttctgtctctggctctcttcggcgacta
cagtggtgtcctcccaatctgcaacggcgacggcaacggcgacagcgccaaaggtactcttctactcgtctcctccccctcctctggtccatacat
ttctttcgatctagttagcgcacacctctcttctttttcccattccttttggttttgaagttgcagagtttgatctgctcagcagaggtgaagagt
tgctgttcggtgttgcggatgctcttcttcagctcctgctctcgattgacccaagtcaggtgatgcaattttttttgtgccgtccgccacaacttc
agcttttgctcatcctgaaaccgattgtgttcctattacagccatgcgtgtagatccagagcgaggggagggtcagatcttgctcgatctgtttct
cccctcctgattaatctcttgtttgctatgctatttttatttctgtgacgcacaaacagaaaatctggttcctggatatttttacttttacactct
agttatattattgtgaaaatgttatctatgtacatcaggaaaagaaaaagaaatataagaaaaataaagcaaagagaaaaaattggtcagtggtca
gagcttagtatatctatccaagctcggaggggtgctgcatcttttttctcttttcactctgtccagcttttccaaaataaataaattagtagtacc
gtgagctgtttctttcacgcacctgaaatgtccagcttttgctcatccggtggtcctctgaaaacaactacagtaggatttttggttattctaatg
tgaatttatgatgtgcagaactggatcttttcttcactagttcaccttggattactgttgcaaaggactctgcccattggatttgaaggagctgtt
attcaaggtactcttcttctactacttctttggaggaaagcaatgcataggtgcatagtgtactgttactttcaccaaccaatcaagttcaggttt
tcctcattctgttttctgaaaaacacaatgttttcattgcagaatttcacgccaaggacaatgctatagccgtctgtagatccagagcaaagacaa
aaaaaggtcgagatatggagatcgccgctggggtgataggccctgtcatccgtaagctcggcgagctgctcgttggagaatacaacctggagaagc
gagtaaagaaaggtgtacaatcgctcctaaacgagctggagatgatgcacgccgtgcttcgcaaggttggcgaggtgccatcggagcagctcgagg
agccagtccggatttgggctggcaaggtgagagacctctcttgcgacatggaagacgctgttgatgacttcctggtgcgtgtggatgagggt
tcaagcagcaagcctacgaacatgaggaatcgagtcaagaagttcctcaagaagaccaccaaactgtttggcaagggcaaagcactt
catcaaatcagtgatgccatcaaagaagctcaggatctcgccaaggagttggcggacctgcgtagaatgtacgagcttgacactcgtag
cactagcaatggtgctaccattgaccctcgtgtgttagctctgcacaaagatgtaggggagcttgttggtgttgaccagacaagggatgagc
ttatcaaaacactgatttgtgaggacgggagttccaaggagcaattgaagacgatctctattgttggtgttggtgggctaggcaagacaacg
atcaccaaagcagtctatgagaagatcaaagcccaatttgattgtgtggcttttgtccccgtgggtcagaacccagatatcaagaaagttttc
aaggacttgctctatggccttgacaatgaaaagttcagtgacattcataatacaacaagggatgaaaatctactcatcaagcaaatcagtg
atttccttgtggataagaggtatgcatgacattgtccttttgtatatattttggaaatgtgtgaagtactgagatgtatactgaatttcatatctt
caatttcttatttgttatatttctatatctgtactatgttctcctttagcaaattctacttctttctttttgcgcacgcgccttatttctgttagt
cctgtatacttttaagcaatttctgttgcatttcttaatctttttgcatgcatatccttagtggttgcataagcctatggtttgctttagtacctt
acagttcatccttgttacaaacttcagttgtatatagattacttgccaactaatgatgtcaaaatatttggcataggtacctgatcgtgatcgatg
atatatgggaagaagaaatatggagatttataaattgtgctttgtataaaaacaaactccatagtcgggtaatcacaacaacccgcaatgtgagtg
tgtctgaagcatgtctctcttccagtgatgacatgattcacaaaatggaacctctttctgatgaagactcgcagatactcttccatcgaagaatat
ttcaaagcgaggacaaatgtccagaagatttgcatgaagtatcaagagagatattgaagaaatgtggtggtgtaccattagccatcattacaatag
ctagccttctagtcagtaaccaaaggataaagcagaaagaagaatggatgcatgtgcacagttcgatgggccttggagttacacaaggtggtattg
tgaaggacatgaagaggatattatcactcagctattatgatttgccatctcacctgaagccttgtttgttatatctaagcatctttcctgaagact
ctgagattaggagagattggctgatatggaggtggcttgctgaagggtttatcctaggtgacacagaagaaactaggctgtttgagatcggagaga
gctacatcaacgagcttatgaacaggagcttgatccagccagcagaaatcaatgaggaaggcacggtagtaactctccgtatacatgatat
ggttcttgatcttatatgctcactgtcaagtgaggagaattttatctccatattagataatgctaagtggcatgcaccttatctgaaaaggaaatt
ccgcaggctatcacttcataatatcaaggcagaggttcagagccatcattttgacagcactagcctgtcaaatgtgaggacctttgctgttttct
ctcctgttacctgtgattggttgccatctctctcaagcttccaatttttacgtgtgctggatcttggaaattgcggcagccgtagcagtagctctg
gtatcggtctcaagtatgtagggaatttaatccacctaaggtacctagggctcaaggatgcagatgtttgcgaactcccaatggacatatgcaa
gttgaagcttttacagacactggatataagaggcaccagtataaaagaattaccttcaagtgttgtacagctcagaaatttgatatgcctatgt
gtctattatacggtgaggctgccaaaaggaatggggagcttgttgtcccttgaagtgctgcaactagtaggcttatcctcatctcctcacattgt
gaaagagctgagccatctgacagaggttaggacactccgtcttgactttgataacatggacgaggatctgattgatatattaatcaagtctct
aggcaaccttcacaaattgcaaaatctgcatattgttgatggtggcagattgatagatcgcatgtgtgaaagctgggtgccccctccaaacc
tccgttgttttgattcatgggacccctctttttcttcgtggttcttgagacttccaaagtgggttaattcaaggtcgcttccccgcctctccaccc
tagaaatagatgtggaagaactgcaaggggatgacattcagatcatcgggatgttgcctgctcttcggtttctgcggctgcgtgcaagtcgcgtg
atgggaacgttggttgtgagggccgatgcattcccatctgcgagatgctgcatgttcagagggtttccgacggcgccatgcctttttccacttg
gagctatgccaagggttcagcgccttcggttctgggtctctgcgcggtcgatcgtgagtggtgaggttgactgcggcatgggccacctccctt
ctctcgaggatgttgaggtttgtctggagcgtgagaattccagcgatgaagagatggagacagccaaggttttgctgaggcgcgcagcag
aagcccatcaaaaacgtcccaccattgaaatctatgacatatgagtgctgacaagcctctgccgccagtaaggtacgtacggtatactcat
ccgaggaaccatgaatcctctttccctgaatacgctcattaccacattgtttgatttatccctcttattacttccccaatttcagggcctcaacaa
aatcctggagtcaggtactagtaattccatcagttgccgtggatgttgttccaatattttgataggcaacaaacaattcattgtgctatcgcagtc
accaataattcactgtaccgccgcaactcccaataaacgaaggctcattccctccaacgtgtgttcagattctgccctttgctagagggaggct
tccgcatgattgttcatgatgaactatttatttctcctgcacctcctttgctttattattcagtcaagactcgcacaataaaaggttgcattcagt
tggcacgatttgtagcagtactgcctctacgtgtatttgattgttcacatacagcatcgttttgtttttactggcttattaattaagagttgattc
aactcctgttggcttgttttgctttgcttggcacttgcttattcagtcaaggctcgtacaatgaactcgtgttgtagctccagatttgattcatcc
tgttcattaacctttctcttcttgctgcaactcctattttcgagctcgtgggagatagagccacaaacgctcactattgatcttcatgacggggaa
atgggagaagtcgcttggctcatgcagcagcagcagcgaagagagcagaagcagatcatgctcaggctgaacagagttggcagagcagaggacgcc
gaagcagagcatgccccagacgctagacacacgcactcctgttgtagctcatttacatcatgttgtttttcaagttttctcttgttgctgcaatta
ttgtttcgagctcgtgatagagagccacaaacgctaagatagaataccataccactattgatcttcatgcctcaaggttacatacacaagtgc
aaaaatgatgaccaaataaaacataagaacaattatataattttgtatgcaaaaagcccacagcagccaaccaccgctcgctcgctcgct
cctgtttcgcataacatcaacggttccaattactgcctctctttcggtttcctttggaacatattttctttctagattatcagttggactaacgaa
atcaattcaatggtgtatttttttctggcaatacatattcaaccgaatcgattatttacaactaacagacaatcaaatagtatgactgagtgagcc
gagtagtagcttgcttacaggattctccaaagctggaaatctacaaagaaatagactcccaaatcctcacaaaggaatgagatcgagaactgc
tacagactccttcgctgttttcacttcctcgtatattcttttgacagatgattgcgtcggcttgtattgcaaacaaatgcgattaggtccgtggcg
atggtggatgattaccagtttaccactattggtctacattctaatattgttgaggttctgctggaaataactgctgcataactagtgactaaaaca
taagaaatctatagcatttctttattgcaaaaaccttagcgcccacaacagccaaccatataactcacccgctctccctctctcggtttttcatta
ggcacatacgttccagataatcaaattgaactgatgacatacagtatgagttacatataaaaaaaattatcactgaaaacttctttcaaataag
gattcaacggtatatatttttgttgttaaacaacacaacctgtcacgttgccttggctgtcacgttgccttggctgccgcatctatctcatgagcc
gcatcaggagtccaaccatattgatagagatagctaggtagttagttcatgtttatctcttgtaacctattctatctcttcttctccaagtcttct
gtaagatctcaactgtatgcgcctcagggttattgcgcccctgcctataacatg.
SEQ ID NO: 2:
atggagatcgccgctggggtgataggccctgtcatccgtaagctcggcgagctgctcgttggagaatacaacctggagaagcgag
taaagaaaggtgtacaatcgctcctaaacgagctggagatgatgcacgccgtgcttcgcaaggttggcgaggtgccatcggagcagctc
gaggagccagtccggatttgggctggcaaggtgagagacctctcttgcgacatggaagacgctgttgatgacttcctggtgcgtgtggatg
agggttcaagcagcaagcctacgaacatgaggaatcgagtcaagaagttcctcaagaagaccaccaaactgtttggcaagggcaaag
cacttcatcaaatcagtgatgccatcaaagaagctcaggatctcgccaaggagttggcggacctgcgtagaatgtacgagcttgacactc
gtagcactagcaatggtgctaccattgaccctcgtgtgttagctctgcacaaagatgtaggggagcttgttggtgttgaccagacaagggat
gagcttatcaaaacactgatttgtgaggacgggagttccaaggagcaattgaagacgatctctattgttggtgttggtgggctaggcaagac
aacgatcaccaaagcagtctatgagaagatcaaagcccaatttgattgtgtggcttttgtccccgtgggtcagaacccagatatcaagaaa
gttttcaaggacttgctctatggccttgacaatgaaaagttcagtgacattcataatacaacaagggatgaaaatctactcatcaagcaaatc
agtgatttccttgtggataagaggtacctgatcgtgatcgatgatatatgggaagaagaaatatggagatttataaattgtgctttgtataaaa
acaaactccatagtcgggtaatcacaacaacccgcaatgtgagtgtgtctgaagcatgtctctcttccagtgatgacatgattcacaaaatg
gaacctctttctgatgaagactcgcagatactcttccatcgaagaatatttcaaagcgaggacaaatgtccagaagatttgcatgaagtatc
aagagagatattgaagaaatgtggtggtgtaccattagccatcattacaatagctagccttctagtcagtaaccaaaggataaagcagaa
agaagaatggatgcatgtgcacagttcgatgggccttggagttacacaaggtggtattgtgaaggacatgaagaggatattatcactcagc
tattatgatttgccatctcacctgaagccttgtttgttatatctaagcatctttcctgaagactctgagattaggagagattggctgatatggagg
tggcttgctgaagggtttatcctaggtgacacagaagaaactaggctgtttgagatcggagagagctacatcaacgagcttatgaacagga
gcttgatccagccagcagaaatcaatgaggaaggcacggtagtaactctccgtatacatgatatggttcttgatcttatatgctcactgtcaa
gtgaggagaattttatctccatattagataatgctaagtggcatgcaccttatctgaaaaggaaattccgcaggctatcacttcataatatcaa
ggcagaggttcagagccatcattttgacagcactagcctgtcaaatgtgaggacctttgctgttttctctcctgttacctgtgattggttgccatc
tctctcaagcttccaatttttacgtgtgctggatcttggaaattgcggcagccgtagcagtagctctggtatcggtctcaagtatgtagggaattt
aatccacctaaggtacctagggctcaaggatgcagatgtttgcgaactcccaatggacatatgcaagttgaagcttttacagacactggatat
aagaggcaccagtataaaagaattaccttcaagtgttgtacagctcagaaatttgatatgcctatgtgtctattatacggtgaggctgccaaa
aggaatggggagcttgttgtcccttgaagtgctgcaactagtaggcttatcctcatctcctcacattgtgaaagagctgagccatctgacaga
ggttaggacactccgtcttgactttgataacatggacgaggatctgattgatatattaatcaagtctctaggcaaccttcacaaattgcaaaat
ctgcatattgttgatggtggcagattgatagatcgcatgtgtgaaagctgggtgccccctccaaacctccgttgttttgattcatgggacccctct
ttttcttcgtggttcttgagacttccaaagtgggttaattcaaggtcgcttccccgcctctccaccctagaaatagatgtggaagaactgcaagg
ggatgacattcagatcatcgggatgttgcctgctcttcggtttctgcggctgcgtgcaagtcgcgtgatgggaacgttggttgtgagggccgat
gcattcccatctgcgagatgctgcatgttcagagggtttccgacggcgccatgcctttttccacttggagctatgccaagggttcagcgccttc
ggttctgggtctctgcgcggtcgatcgtgagtggtgaggttgactgcggcatgggccacctcccttctctcgaggatgttgaggtttgtctgga
gcgtgagaattccagcgatgaagagatggagacagccaaggttttgctgaggcgcgcagcagaagcccatcaaaaacgtcccaccat
tgaaatctatgacatatga.
SEQ ID NO: 3:
MEIAAGVIGPVIRKLGELLVGEYNLEKRVKKGVQSLLNELEMMHAVLRKVGEVPSEQLEEPVRI
WAGKVRDLSCDMEDAVDDFLVRVDEGSSSKPTNMRNRVKKFLKKTTKLFGKGKALHQISDAIKEA
QDLAKELADLRRMYELDTRSTSNGATIDPRVLALHKDVGELVGVDQTRDELIKTLICEDGSSKEQL
KTISIVGVGGLGKTTITKAVYEKIKAQFDCVAFVPVGQNPDIKKVFKDLLYGLDNEKFSDIHNTTRDE
NLLIKQISDFLVDKRYLIVIDDIWEEEIWRFINCALYKNKLHSRVITTTRNVSVSEACLSSSDDMIHKM
EPLSDEDSQILFHRRIFQSEDKCPEDLHEVSREILKKCGGVPLAIITIASLLVSNQRIKQKEEWMHVH
SSMGLGVTQGGIVKDMKRILSLSYYDLPSHLKPCLLYLSIFPEDSEIRRDWLIWRWLAEGFILGDTE
ETRLFEIGESYINELMNRSLIQPAEINEEGTVVTLRIHDMVLDLICSLSSEENFISILDNAKWHAPYLK
RKFRRLSLHNIKAEVQSHHFDSTSLSNVRTFAVFSPVTCDWLPSLSSFQFLRVLDLGNCGSRSSSS
GIGLKYVGNLIHLRYLGLKDADVCELPMDICKLKLLQTLDIRGTSIKELPSSVVQLRNLICLCVYYTVR
LPKGMGSLLSLEVLQLVGLSSSPHIVKELSHLTEVRTLRLDFDNMDEDLIDILIKSLGNLHKLQNLHIV
DGGRLIDRMCESWVPPPNLRCFDSWDPSFSSWFLRLPKWVNSRSLPRLSTLEIDVEELQGDDIQII
GMLPALRFLRLRASRVMGTLVVRADAFPSARCCMFRGFPTAPCLFPLGAMPRVQRLRFWVSARSI
VSGEVDCGMGHLPSLEDVEVCLERENSSDEEMETAKVLLRRAAEAHQKRPTIEIYDI.
p1300-Lr47F1: (SEQ ID NO: 4):
ccatgattacgaattcgagctccctcggtgaagttggcgtcgaagt.
p1300-Lr47R1: (SEQ ID NO: 5):
tcatgtccttcacaataccaccttgtgtaact.
p1300-Lr47F2: (SEQ ID NO: 6):
ggacatgaagaggatattatcactcagctattatgatttgcc.
p1300-Lr47R2: (SEQ ID NO: 7):
acggccagtgccaagcttcatgttataggcaggggcgc.

### 2. Acquisition of to Generation Transgenic Plants

The p1300-Lr47 complementary vector plasmid was extracted and purified using a plasmid extraction kit (TIANGEN BIOTECH (BEIJING) CO., LTD.). The plasmid was then introduced into the Agrobacterium strain EHA105 and was transferred into the common wheat cultivar Fielder using the Agrobacterium-mediated transformation method, and thus 80 To generation Lr47 complementation transgenic plants were obtained.

### 3. Identification of Resistance of Transgenic Plants (Family)

Firstly, the obtained T₀ generation complementary transgenic plants were screened using a marker to confirm successful transformation, and the results indicated that the 80 transgenic plants tested positive. The T₀ transgenic plants were grown in a greenhouse and were selfed to produce T₁ generation seeds. The Ti transgenic families were inoculated with Pt pathotype PHQS, and they were phenotypically assessed 10 days after inoculation. As shown in (b) in FIG. 4, the T₁ generation transgenic plants exhibited near-immune resistant phenotypes (2, 3, 4, 5, 6, and 7), while the control Fielder (1) exhibited high susceptibility.

From the description above, it can be determined that the described embodiments of the present application achieve the following technical effects: The present application provides the leaf rust resistance gene Lr47, which is useful for studying the disease resistance mechanisms of resistance genes against pathogens; the nucleotide sequence encoding the Lr47 protein is introduced into wheat, which can improve the resistance of wheat to leaf rust and provide a reliable and effective leaf rust resistance source for wheat molecular breeding. The Lr47 protein confers wheat leaf rust resistance, addressing the problem of wheat susceptibility to leaf rust and associated yield losses. This has applications in wheat breeding and biotechnology.

The description above constitutes only the preferred embodiments of the present application and is not intended to limit the scope of the present application. For a person skilled in the art can make various modifications and variations to the present application. Any modifications, equivalent replacements, improvements, and the like made within the spirit and principle of the present application shall fall within the scope of protection of the present application.

1. A wheat leaf rust resistance protein, wherein the wheat leaf rust resistance protein is as shown in any one of the following (a)-(c):
(a) a protein having an amino acid sequence as shown in SEQ ID NO: 3; or
(b) a protein, which is obtained by substituting and/or deleting and/or adding one or more amino acids in the amino acid sequence of (a) and which has anti-wheat leaf rust activity; or
(c) a protein having at least 80% or more sequence identity with the amino acid sequence as defined in any one of (a) or (b) and having the same function.

2. The wheat leaf rust resistance protein of claim 1, wherein the wheat leaf rust resistance protein is a protein having 85% or more, preferably 90% or more, more preferably 95% or more, and even more preferably 99% or more sequence identity with the amino acid sequence as defined in any one of (a) or (b) and having the same function.

3. A wheat leaf rust resistance gene, wherein the wheat leaf rust resistance gene is as shown in any one of the following (i)-(iv):
(i) a nucleotide sequence encoding the wheat leaf rust resistance protein of claim 1; or
(ii) a nucleotide sequence that hybridizes under stringent conditions to the DNA molecule defined in (i) and encodes the wheat leaf rust resistance protein of claim 1; or
(iii) a nucleotide sequence as shown in SEQ ID NO: 2; or
(iv) a gene having 70% or more identity with any one of the nucleotide sequences defined in (i)-(iii) and encoding a protein having the same function.

4. The wheat leaf rust resistance gene of claim 3, wherein the wheat leaf rust resistance gene is a gene having 75% or more, preferably 85% or more, more preferably 95% or more, and even more preferably 99% or more identity with any one of the nucleotide sequences as defined in (i)-(iii) and encoding a protein having the same function.

5. An expression cassette, wherein the expression cassette comprises a regulatory sequence and the wheat leaf rust resistance gene of claim 3 or a gene having 75% or more, preferably 85% or more, more preferably 95% or more, and even more preferably 99% or more identity with any one of the nucleotide sequences as defined in (i)-(iii) and encoding a protein having the same function.

6. The expression cassette of claim 5, wherein the regulatory sequence comprises a promoter; and preferably, the promoter comprises one or more of the following promoters: constitutive promoters, enhanced promoters, tissue specific promoters, or inducible promoters.

7. A recombinant vector comprising the wheat leaf rust resistance gene of claim 3 or a gene having 75% or more, preferably 85% or more, more preferably 95% or more, and even more preferably 99% or more identity with any of the nucleotide sequences as defined in (a) to (c) and encoding a protein having the same function or the expression cassette comprising a regulatory sequence and the wheat leaf rust resistance gene;
preferably, the regulatory sequence comprises a promoter;
more preferably, the promoter comprises one or more of the following promoters: constitutive promoters, enhanced promoters, tissue specific promoters or inducible promoters.

8. The recombinant vector of claim 7, wherein the recombinant vector comprises a translational control signal;
preferably, the translational control signal comprises an enhancer;
preferably, the translational control signal comprises an enhancer;
preferably, the enhancer comprises a translational enhancer and/or a transcriptional enhancer;
preferably, the translational control signal is derived from a natural sequence or an artificial synthetic sequence;
preferably, the recombinant vector comprises a plant expression vector;
preferably, the plant expression vector comprises a binary vector for Agrobacterium transformation and a gene gun bombardment vector;
preferably, the plant expression vector comprises pCAMBIA1300;
preferably, the recombinant vector comprises a reporter gene;
preferably, the reporter gene comprises a resistance gene or a gene expressing an enzyme or a luminescent compound producing a color change; and
preferably, the resistance gene comprises an antibiotic resistance gene or a chemical agent resistance gene.

9. A host cell transformed with the recombinant vector of claim 7;
preferably, the host cell comprises a non-plant host cell;
preferably, the host cell comprises Escherichia coli or Agrobacterium tumefaciens;
preferably, the Escherichia coli comprises DH5a; and
preferably, the Agrobacterium tumefaciens comprises EHA105.

10. Use of the wheat leaf rust resistance protein of claim 1 or the wheat leaf rust resistance protein having 85% or more, preferably 90% or more, more preferably 95% or more, and even more preferably 99% or more identity with the amino acid sequence as defined in any one of (a) or (b) and having the same function; or
a wheat leaf rust resistance gene which is as shown in any one of the following (i)-(iv):
(i) a nucleotide sequence encoding the wheat leaf rust resistance protein of claim 1; or
(ii) a nucleotide sequence that hybridizes under stringent conditions to the DNA molecule defined in (a) and encodes the wheat leaf rust resistance protein of claim 1; or
(iii) a nucleotide sequence as shown in SEQ ID NO: 2; or
(iv) a gene having 70% or more, preferably having 75% or more, preferably 85% or more, more preferably 95% or more, and even more preferably 99% or more identity with any one of the nucleotide sequences defined in (i)-(iii) and encoding a protein having the same function; or
an expression cassette comprising a regulatory sequence and the wheat leaf rust resistance gene;
preferably, the expression cassette comprises a promoter; and
more preferably, the promoter comprises one or more of the following promoters: constitutive promoters, enhanced promoters, tissue specific promoters, or inducible promoters; or
a recombinant vector comprising the wheat leaf rust resistance gene or the expression cassette;
preferably, the recombinant vector comprises a translational control signal;
preferably, the translational control signal comprises an enhancer;
preferably, the enhancer comprises a translational enhancer and/or a transcriptional enhancer;
preferably, the translational control signal is derived from a natural sequence or an artificial synthetic sequence;
preferably, the recombinant vector comprises a plant expression vector;
preferably, the plant expression vector comprises a binary vector for Agrobacterium-mediated transformation or a gene gun bombardment vector;
preferably, the plant expression vector comprises pCAMBIA1300;
preferably, the recombinant vector comprises a reporter gene;
preferably, the reporter gene comprises a resistance gene, or a gene expressing an enzyme or a luminescent compound producing a color change; and
preferably, the resistance gene comprises an antibiotic resistance gene or a chemical agent resistance gene; or
a host cell transformed with the recombinant vector,
preferably, the host cell comprises a non-plant host cell;
preferably, the host cell comprises Escherichia coli or Agrobacterium tumefaciens;
preferably, the Escherichia coli comprises DH5α; and
preferably, the Agrobacterium tumefaciens comprises EHA105;
for use in regulating the resistance of a plant to leaf rust, enhancing or reducing the resistance of the plant to leaf rust, or culturing a transgenic plant having enhanced or reduced resistance to leaf rust, or wheat breeding for resistance to leaf rust.

11. A method for preparing a transgenic plant, comprising:
introducing the wheat leaf rust resistance gene of claim 3 or a gene having 75% or more, preferably 85% or more, more preferably 95% or more, and even more preferably 99% or more identity with any of the nucleotide sequences as defined in (a) to (c) and encoding a protein having the same function;
or an expression cassette comprising a regulatory sequence and the wheat leaf rust resistance gene;
preferably, the expression cassette comprises a promoter; and
more preferably, the promoter comprises one or more of the following promoters:
constitutive promoters, enhanced promoters, tissue specific promoters, or inducible promoters;
or a recombinant vector comprising the wheat leaf rust resistance gene or the expression cassette;
preferably, the recombinant vector comprises a translational control signal;
preferably, the translational control signal comprises an enhancer;
preferably, the enhancer comprises a translational enhancer and/or a transcriptional enhancer;
preferably, the translational control signal is derived from a natural sequence or an artificial synthetic sequence;
preferably, the recombinant vector comprises a plant expression vector;
preferably, the plant expression vector comprises a binary vector for Agrobacterium-mediated transformation or a gene gun bombardment vector;
preferably, the plant expression vector comprises pCAMBIA1300;
preferably, the recombinant vector comprises a reporter gene;
preferably, the reporter gene comprises a resistance gene, or a gene expressing an enzyme or a luminescent compound producing a color change; and
preferably, the resistance gene comprises an antibiotic resistance gene or a chemical agent resistance gene;
or a host cell transformed with the recombinant vector;
preferably, the host cell comprises a non-plant host cell;
preferably, the host cell comprises Escherichia coli or Agrobacterium tumefaciens;
preferably, the Escherichia coli comprises DH5α; and
preferably, the Agrobacterium tumefaciens comprises EHA105;
into a target plant to obtain the transgenic plant resistant to leaf rust.

12. The method of claim 11, wherein the recombinant vector is introduced into the target plant by methods comprising a plant viral vector, a gene gun, or Agrobacterium-mediated transformation;
preferably, the target plant is a dicotyledonous plant or a monocotyledonous plant;
preferably, the target plant is wheat;
preferably, the wheat is Fielder wheat; and
preferably, the wheat leaf rust resistance gene is driven by a constitutive promoter.

13. A breeding method for enhancing or reducing the resistance of a plant to leaf rust, wherein the breeding method comprises:
enhancing or reducing the activity or amount of the wheat leaf rust resistance protein of claim 1 or the wheat leaf rust resistance protein having 85% or more, preferably 90% or more, more preferably 95% or more, and even more preferably 99% or more identity with the amino acid sequence as defined in any one of (a) or (b) and having the same function in a target plant to enhance or reduce the resistance of the plant to resistance.

14. The breeding method of claim 13, wherein the target plant is a dicotyledonous plant or a monocotyledonous plant;
preferably, the target plant is wheat;
preferably, the wheat is Fielder wheat;
preferably, the leaf rust is leaf rust caused by a Pt pathotype; and
preferably, the Pt pathotype is a Pt pathotype prevalent in China, and the Pt pathotype prevalent in China comprises FHJL, PHQS, FHJR, THDB, PHRT, PHTT, THTT, HCJR, or FHHM.

15. A transgenic wheat plant, wherein the transgenic wheat plant comprises a gene that is as shown in any one of the following (i)-(iv):
(i) a nucleotide sequence encoding the wheat leaf rust resistance protein of claim 1; or
(ii) a nucleotide sequence that hybridizes under stringent conditions to the DNA molecule defined in (i) and encodes the wheat leaf rust resistance protein of claim 1; or
(iii) a nucleotide sequence as shown in SEQ ID NO: 2; or
(iv) a gene having 70% or more identity with any one of the nucleotide sequences defined in (i)-(iii) and encoding a protein having the same function.


SEQUENCE LISTING (7 sequences)

SEQ ID NO: 1 - DNA, 7234 bp, Triticum aestivum
  source 1..7234: mol_type = genomic DNA
cctcggtgaagttggcgtcgaagtgtggctgatggtgcgggtcaggttcatctgtgtggcagtccctaggtgcgggtgaaggccatcacgacggatctgatccccgcatcgattggtggttgctgcagttcctggtgtgctgaaggtggtggctggaggcgtgcttcctcggcagtgatgtgtggcttcgactgcaagtggctgcggtgttggtggtgagaggcatctttcggggctggccggaagcttggtggcgtggaaatgtgcaatgctacggatgaaaattctgttcggccttggtcggaccggcatcgatggcacccgtgggtgtcattccccttcctggaggcgccgccgagggtagcgccattgtccacgttgccttgtatcggcaaccatctccggggcgaaagccttgatccgtaggatcggcacgacggcggcgtctttctgatgttgtttctctgttgggagcttcgtgcttggagataagaggccctatgttgcgctcctccggcgtgcaccgctacccggatcgttcttctctggcgctatgtacgatcgtcgctggttctaccaaggaagctggagttgctgttcttcgatgatctccggcgtcggtcgagacgcggcgaggatatcggttttgggtaggctcttgtatgtcgtagtgtattgtcgtgtgtggtttcctttgtttgtgtcaggtgtggagttgtgctacgctcgttgttcgcagcgagaggtagatgtggttgtatggctgtatttctctccttctataaagctaaggtatgcattttgcgtaccctcgaaaaaaacacgttagtggaggctgttgctgctaatacgttagtggaggctgctttttgttttctttttttttctttttctttttgagggggttagtggagctgctgctgctaatgcgcggagagatgcgaggagacgaccgcaagtcgttcattccatttccaggtccacaaatagtttgtttccttctgtctctggctctcttcggcgactacagtggtgtcctcccaatctgcaacggcgacggcaacggcgacagcgccaaaggtactcttctactcgtctcctccccctcctctggtccatacatttctttcgatctagttagcgcacacctctcttctttttcccattccttttggttttgaagttgcagagtttgatctgctcagcagaggtgaagagttgctgttcggtgttgcggatgctcttcttcagctcctgctctcgattgacccaagtcaggtgatgcaattttttttgtgccgtccgccacaacttcagcttttgctcatcctgaaaccgattgtgttcctattacagccatgcgtgtagatccagagcgaggggagggtcagatcttgctcgatctgtttctcccctcctgattaatctcttgtttgctatgctatttttatttctgtgacgcacaaacagaaaatctggttcctggatatttttacttttacactctagttatattattgtgaaaatgttatctatgtacatcaggaaaagaaaaagaaatataagaaaaataaagcaaagagaaaaaattggtcagtggtcagagcttagtatatctatccaagctcggaggggtgctgcatcttttttctcttttcactctgtccagcttttccaaaataaataaattagtagtaccgtgagctgtttctttcacgcacctgaaatgtccagcttttgctcatccggtggtcctctgaaaacaactacagtaggatttttggttattctaatgtgaatttatgatgtgcagaactggatcttttcttcactagttcaccttggattactgttgcaaaggactctgcccattggatttgaaggagctgttattcaaggtactcttcttctactacttctttggaggaaagcaatgcataggtgcatagtgtactgttactttcaccaaccaatcaagttcaggttttcctcattctgttttctgaaaaacacaatgttttcattgcagaatttcacgccaaggacaatgctatagccgtctgtagatccagagcaaagacaaaaaaaggtcgagatatggagatcgccgctggggtgataggccctgtcatccgtaagctcggcgagctgctcgttggagaatacaacctggagaagcgagtaaagaaaggtgtacaatcgctcctaaacgagctggagatgatgcacgccgtgcttcgcaaggttggcgaggtgccatcggagcagctcgaggagccagtccggatttgggctggcaaggtgagagacctctcttgcgacatggaagacgctgttgatgacttcctggtgcgtgtggatgagggttcaagcagcaagcctacgaacatgaggaatcgagtcaagaagttcctcaagaagaccaccaaactgtttggcaagggcaaagcacttcatcaaatcagtgatgccatcaaagaagctcaggatctcgccaaggagttggcggacctgcgtagaatgtacgagcttgacactcgtagcactagcaatggtgctaccattgaccctcgtgtgttagctctgcacaaagatgtaggggagcttgttggtgttgaccagacaagggatgagcttatcaaaacactgatttgtgaggacgggagttccaaggagcaattgaagacgatctctattgttggtgttggtgggctaggcaagacaacgatcaccaaagcagtctatgagaagatcaaagcccaatttgattgtgtggcttttgtccccgtgggtcagaacccagatatcaagaaagttttcaaggacttgctctatggccttgacaatgaaaagttcagtgacattcataatacaacaagggatgaaaatctactcatcaagcaaatcagtgatttccttgtggataagaggtatgcatgacattgtccttttgtatatattttggaaatgtgtgaagtactgagatgtatactgaatttcatatcttcaatttcttatttgttatatttctatatctgtactatgttctcctttagcaaattctacttctttctttttgcgcacgcgccttatttctgttagtcctgtatacttttaagcaatttctgttgcatttcttaatctttttgcatgcatatccttagtggttgcataagcctatggtttgctttagtaccttacagttcatccttgttacaaacttcagttgtatatagattacttgccaactaatgatgtcaaaatatttggcataggtacctgatcgtgatcgatgatatatgggaagaagaaatatggagatttataaattgtgctttgtataaaaacaaactccatagtcgggtaatcacaacaacccgcaatgtgagtgtgtctgaagcatgtctctcttccagtgatgacatgattcacaaaatggaacctctttctgatgaagactcgcagatactcttccatcgaagaatatttcaaagcgaggacaaatgtccagaagatttgcatgaagtatcaagagagatattgaagaaatgtggtggtgtaccattagccatcattacaatagctagccttctagtcagtaaccaaaggataaagcagaaagaagaatggatgcatgtgcacagttcgatgggccttggagttacacaaggtggtattgtgaaggacatgaagaggatattatcactcagctattatgatttgccatctcacctgaagccttgtttgttatatctaagcatctttcctgaagactctgagattaggagagattggctgatatggaggtggcttgctgaagggtttatcctaggtgacacagaagaaactaggctgtttgagatcggagagagctacatcaacgagcttatgaacaggagcttgatccagccagcagaaatcaatgaggaaggcacggtagtaactctccgtatacatgatatggttcttgatcttatatgctcactgtcaagtgaggagaattttatctccatattagataatgctaagtggcatgcaccttatctgaaaaggaaattccgcaggctatcacttcataatatcaaggcagaggttcagagccatcattttgacagcactagcctgtcaaatgtgaggacctttgctgttttctctcctgttacctgtgattggttgccatctctctcaagcttccaatttttacgtgtgctggatcttggaaattgcggcagccgtagcagtagctctggtatcggtctcaagtatgtagggaatttaatccacctaaggtacctagggctcaaggatgcagatgtttgcgaactcccaatggacatatgcaagttgaagcttttacagacactggatataagaggcaccagtataaaagaattaccttcaagtgttgtacagctcagaaatttgatatgcctatgtgtctattatacggtgaggctgccaaaaggaatggggagcttgttgtcccttgaagtgctgcaactagtaggcttatcctcatctcctcacattgtgaaagagctgagccatctgacagaggttaggacactccgtcttgactttgataacatggacgaggatctgattgatatattaatcaagtctctaggcaaccttcacaaattgcaaaatctgcatattgttgatggtggcagattgatagatcgcatgtgtgaaagctgggtgccccctccaaacctccgttgttttgattcatgggacccctctttttcttcgtggttcttgagacttccaaagtgggttaattcaaggtcgcttccccgcctctccaccctagaaatagatgtggaagaactgcaaggggatgacattcagatcatcgggatgttgcctgctcttcggtttctgcggctgcgtgcaagtcgcgtgatgggaacgttggttgtgagggccgatgcattcccatctgcgagatgctgcatgttcagagggtttccgacggcgccatgcctttttccacttggagctatgccaagggttcagcgccttcggttctgggtctctgcgcggtcgatcgtgagtggtgaggttgactgcggcatgggccacctcccttctctcgaggatgttgaggtttgtctggagcgtgagaattccagcgatgaagagatggagacagccaaggttttgctgaggcgcgcagcagaagcccatcaaaaacgtcccaccattgaaatctatgacatatgagtgctgacaagcctctgccgccagtaaggtacgtacggtatactcatccgaggaaccatgaatcctctttccctgaatacgctcattaccacattgtttgatttatccctcttattacttccccaatttcagggcctcaacaaaatcctggagtcaggtactagtaattccatcagttgccgtggatgttgttccaatattttgataggcaacaaacaattcattgtgctatcgcagtcaccaataattcactgtaccgccgcaactcccaataaacgaaggctcattccctccaacgtgtgttcagattctgccctttgctagagggaggcttccgcatgattgttcatgatgaactatttatttctcctgcacctcctttgctttattattcagtcaagactcgcacaataaaaggttgcattcagttggcacgatttgtagcagtactgcctctacgtgtatttgattgttcacatacagcatcgttttgtttttactggcttattaattaagagttgattcaactcctgttggcttgttttgctttgcttggcacttgcttattcagtcaaggctcgtacaatgaactcgtgttgtagctccagatttgattcatcctgttcattaacctttctcttcttgctgcaactcctattttcgagctcgtgggagatagagccacaaacgctcactattgatcttcatgacggggaaatgggagaagtcgcttggctcatgcagcagcagcagcgaagagagcagaagcagatcatgctcaggctgaacagagttggcagagcagaggacgccgaagcagagcatgccccagacgctagacacacgcactcctgttgtagctcatttacatcatgttgtttttcaagttttctcttgttgctgcaattattgtttcgagctcgtgatagagagccacaaacgctaagatagaataccataccactattgatcttcatgcctcaaggttacatacacaagtgcaaaaatgatgaccaaataaaacataagaacaattatataattttgtatgcaaaaagcccacagcagccaaccaccgctcgctcgctcgctcctgtttcgcataacatcaacggttccaattactgcctctctttcggtttcctttggaacatattttctttctagattatcagttggactaacgaaatcaattcaatggtgtatttttttctggcaatacatattcaaccgaatcgattatttacaactaacagacaatcaaatagtatgactgagtgagccgagtagtagcttgcttacaggattctccaaagctggaaatctacaaagaaatagactcccaaatcctcacaaaggaatgagatcgagaactgctacagactccttcgctgttttcacttcctcgtatattcttttgacagatgattgcgtcggcttgtattgcaaacaaatgcgattaggtccgtggcgatggtggatgattaccagtttaccactattggtctacattctaatattgttgaggttctgctggaaataactgctgcataactagtgactaaaacataagaaatctatagcatttctttattgcaaaaaccttagcgcccacaacagccaaccatataactcacccgctctccctctctcggtttttcattaggcacatacgttccagataatcaaattgaactgatgacatacagtatgagttacatataaaaaaaattatcactgaaaacttctttcaaataaggattcaacggtatatatttttgttgttaaacaacacaacctgtcacgttgccttggctgtcacgttgccttggctgccgcatctatctcatgagccgcatcaggagtccaaccatattgatagagatagctaggtagttagttcatgtttatctcttgtaacctattctatctcttcttctccaagtcttctgtaagatctcaactgtatgcgcctcagggttattgcgcccctgcctataacatg

SEQ ID NO: 2 - DNA, 2787 bp, Triticum aestivum
  source 1..2787: mol_type = genomic DNA
atggagatcgccgctggggtgataggccctgtcatccgtaagctcggcgagctgctcgttggagaatacaacctggagaagcgagtaaagaaaggtgtacaatcgctcctaaacgagctggagatgatgcacgccgtgcttcgcaaggttggcgaggtgccatcggagcagctcgaggagccagtccggatttgggctggcaaggtgagagacctctcttgcgacatggaagacgctgttgatgacttcctggtgcgtgtggatgagggttcaagcagcaagcctacgaacatgaggaatcgagtcaagaagttcctcaagaagaccaccaaactgtttggcaagggcaaagcacttcatcaaatcagtgatgccatcaaagaagctcaggatctcgccaaggagttggcggacctgcgtagaatgtacgagcttgacactcgtagcactagcaatggtgctaccattgaccctcgtgtgttagctctgcacaaagatgtaggggagcttgttggtgttgaccagacaagggatgagcttatcaaaacactgatttgtgaggacgggagttccaaggagcaattgaagacgatctctattgttggtgttggtgggctaggcaagacaacgatcaccaaagcagtctatgagaagatcaaagcccaatttgattgtgtggcttttgtccccgtgggtcagaacccagatatcaagaaagttttcaaggacttgctctatggccttgacaatgaaaagttcagtgacattcataatacaacaagggatgaaaatctactcatcaagcaaatcagtgatttccttgtggataagaggtacctgatcgtgatcgatgatatatgggaagaagaaatatggagatttataaattgtgctttgtataaaaacaaactccatagtcgggtaatcacaacaacccgcaatgtgagtgtgtctgaagcatgtctctcttccagtgatgacatgattcacaaaatggaacctctttctgatgaagactcgcagatactcttccatcgaagaatatttcaaagcgaggacaaatgtccagaagatttgcatgaagtatcaagagagatattgaagaaatgtggtggtgtaccattagccatcattacaatagctagccttctagtcagtaaccaaaggataaagcagaaagaagaatggatgcatgtgcacagttcgatgggccttggagttacacaaggtggtattgtgaaggacatgaagaggatattatcactcagctattatgatttgccatctcacctgaagccttgtttgttatatctaagcatctttcctgaagactctgagattaggagagattggctgatatggaggtggcttgctgaagggtttatcctaggtgacacagaagaaactaggctgtttgagatcggagagagctacatcaacgagcttatgaacaggagcttgatccagccagcagaaatcaatgaggaaggcacggtagtaactctccgtatacatgatatggttcttgatcttatatgctcactgtcaagtgaggagaattttatctccatattagataatgctaagtggcatgcaccttatctgaaaaggaaattccgcaggctatcacttcataatatcaaggcagaggttcagagccatcattttgacagcactagcctgtcaaatgtgaggacctttgctgttttctctcctgttacctgtgattggttgccatctctctcaagcttccaatttttacgtgtgctggatcttggaaattgcggcagccgtagcagtagctctggtatcggtctcaagtatgtagggaatttaatccacctaaggtacctagggctcaaggatgcagatgtttgcgaactcccaatggacatatgcaagttgaagcttttacagacactggatataagaggcaccagtataaaagaattaccttcaagtgttgtacagctcagaaatttgatatgcctatgtgtctattatacggtgaggctgccaaaaggaatggggagcttgttgtcccttgaagtgctgcaactagtaggcttatcctcatctcctcacattgtgaaagagctgagccatctgacagaggttaggacactccgtcttgactttgataacatggacgaggatctgattgatatattaatcaagtctctaggcaaccttcacaaattgcaaaatctgcatattgttgatggtggcagattgatagatcgcatgtgtgaaagctgggtgccccctccaaacctccgttgttttgattcatgggacccctctttttcttcgtggttcttgagacttccaaagtgggttaattcaaggtcgcttccccgcctctccaccctagaaatagatgtggaagaactgcaaggggatgacattcagatcatcgggatgttgcctgctcttcggtttctgcggctgcgtgcaagtcgcgtgatgggaacgttggttgtgagggccgatgcattcccatctgcgagatgctgcatgttcagagggtttccgacggcgccatgcctttttccacttggagctatgccaagggttcagcgccttcggttctgggtctctgcgcggtcgatcgtgagtggtgaggttgactgcggcatgggccacctcccttctctcgaggatgttgaggtttgtctggagcgtgagaattccagcgatgaagagatggagacagccaaggttttgctgaggcgcgcagcagaagcccatcaaaaacgtcccaccattgaaatctatgacatatga

SEQ ID NO: 3 - protein, 928 aa, Triticum aestivum
MEIAAGVIGPVIRKLGELLVGEYNLEKRVKKGVQSLLNELEMMHAVLRKVGEVPSEQLEEPVRIWAGKVRDLSCDMEDAVDDFLVRVDEGSSSKPTNMRNRVKKFLKKTTKLFGKGKALHQISDAIKEAQDLAKELADLRRMYELDTRSTSNGATIDPRVLALHKDVGELVGVDQTRDELIKTLICEDGSSKEQLKTISIVGVGGLGKTTITKAVYEKIKAQFDCVAFVPVGQNPDIKKVFKDLLYGLDNEKFSDIHNTTRDENLLIKQISDFLVDKRYLIVIDDIWEEEIWRFINCALYKNKLHSRVITTTRNVSVSEACLSSSDDMIHKMEPLSDEDSQILFHRRIFQSEDKCPEDLHEVSREILKKCGGVPLAIITIASLLVSNQRIKQKEEWMHVHSSMGLGVTQGGIVKDMKRILSLSYYDLPSHLKPCLLYLSIFPEDSEIRRDWLIWRWLAEGFILGDTEETRLFEIGESYINELMNRSLIQPAEINEEGTVVTLRIHDMVLDLICSLSSEENFISILDNAKWHAPYLKRKFRRLSLHNIKAEVQSHHFDSTSLSNVRTFAVFSPVTCDWLPSLSSFQFLRVLDLGNCGSRSSSSGIGLKYVGNLIHLRYLGLKDADVCELPMDICKLKLLQTLDIRGTSIKELPSSVVQLRNLICLCVYYTVRLPKGMGSLLSLEVLQLVGLSSSPHIVKELSHLTEVRTLRLDFDNMDEDLIDILIKSLGNLHKLQNLHIVDGGRLIDRMCESWVPPPNLRCFDSWDPSFSSWFLRLPKWVNSRSLPRLSTLEIDVEELQGDDIQIIGMLPALRFLRLRASRVMGTLVVRADAFPSARCCMFRGFPTAPCLFPLGAMPRVQRLRFWVSARSIVSGEVDCGMGHLPSLEDVEVCLERENSSDEEMETAKVLLRRAAEAHQKRPTIEIYDI

SEQ ID NO: 4 - DNA, 46 bp, synthetic construct
  source 1..46: mol_type = other DNA; note = p1300-Lr47F1
ccatgattacgaattcgagctccctcggtgaagttggcgtcgaagt

SEQ ID NO: 5 - DNA, 32 bp, synthetic construct
  source 1..32: mol_type = other DNA; note = p1300-Lr47R1
tcatgtccttcacaataccaccttgtgtaact

SEQ ID NO: 6 - DNA, 42 bp, synthetic construct
  source 1..42: mol_type = other DNA; note = p1300-Lr47F2
ggacatgaagaggatattatcactcagctattatgatttgcc

SEQ ID NO: 7 - DNA, 38 bp, synthetic construct
  source 1..38: mol_type = other DNA; note = p1300-Lr47R2
acggccagtgccaagcttcatgttataggcaggggcgc